(19) United States
(12) Patent Application Publication
(10) Pub. No.: US 2017/0007793 A1
(43) Pub. Date: Jan. 12, 2017

(54) Non-Rebreather Face Mask

(71) Applicants: Chris SALVINO, Phoenix, AZ (US); Scott WHITE, Phoenix, AZ (US)

(72) Inventors: Chris SALVINO, Phoenix, AZ (US); Scott WHITE, Phoenix, AZ (US)

(21) Appl. No.: 15/122,119

(22) PCT Filed: Feb. 27, 2015
(86) PCT No.: PCT/US15/18021

§ 371 Date: Aug. 26, 2016

Related U.S. Application Data
(60) Provisional application No. 61/946,602, filed on Feb. 28, 2014.

(51) Int. Cl. A61M 16/06; A61M 16/08; A61M 16/00; A61M 16/20

(52) U.S. Cl. CPC A61M 16/0605; A61M 16/208; A61M 16/0816; A61M 16/0078; A61M 16/0875

(57) ABSTRACT

The invention is an improved non-rebreather face mask (NRB). The mask enables users to obtain high purity oxygen, over 80%, while minimizing the amount of pure oxygen needed to run the system by reducing dead space within the mask. The mask also features safety mechanisms to ensure users will never suffocate.

### CROSS-REFERENCE TO RELATED APPLICATION

This application claims priority to PCT application PCT/2015US/018021 filed Feb. 27, 2015 and to U.S. provisional application No. 61/946,602 filed Feb. 28, 2014 the contents of which are hereby incorporated by reference.

### BACKGROUND

The present invention relates to the medical field and to oxygen supplying face masks. Non-rebreather face masks are used in medical emergencies that require oxygen therapy. Such emergencies include physical trauma, chronic airway limitation, chronic obstructive pulmonary diseases, cluster headaches, smoke inhalation, and carbon monoxide poisoning.

The mask covers both the nose and the mouth of the patient. The mask is attached to patients by an elastic cord that wraps from either side of the mask around the patient's head. The NRB has an attached reservoir bag that is usually 1 liter in volume that connects to an external tank or other oxygen supply system. To place an NRB on a patient, the reservoir bag must be inflated to greater than ⅔ full, at the rate of 15 liters per minute. When a patient inhales, approximately ⅓ the volume of 80% pure oxygen in the reservoir is removed from the reservoir bag. This volume of mixed air and oxygen is replaced by flow from the oxygen supply.

Typical NRB masks are designed to capture the first 150 ml of exhaled breath. This captured air is held in the reservoir bag and is inhaled during the subsequent breath. However, this 150 ml of low oxygen concentration air is delivered at the end of the following inhalation and is therefore delivered to the dead space anatomy where gas exchange does not occur, and therefore does not result in depletion of oxygen during the rebreathing step. The rest of the exhaled air is directed through a check valve that allows spent air to only pass out of the mask. The check valve prevents the inhalation of atmospheric room air. NRB masks that are currently available allow for administration of purified oxygen at concentrations of between 60-80% purity. Due to the risk of suffocation that may occur from a malfunction in the mask, at least one of the check valves is often removed during operation. While this enables a user to breathe air from outside the system in the event of a failure in the gas supply, this added safety precaution results in a much lower purity of oxygen being delivered to the patient and reduces the effectiveness of NRB mask for patients who need them.

The major problems with prior versions of the NRB masks therefore are relatively low oxygen concentration and safety concerns. Delivery of high purity oxygen to patients is a difficult clinical problem.

The present invention allows the face mask to fit snugly on the face of patients without leakage and this further enables high purity oxygen to enter the body due to two improvements, an elastic reservoir bag and an inner inflatable lining of the mask.

### RELEVANT ART REFERENCES

US2003/0024533—Sniadach—Multi-Port Mask—discloses a nose and mouth mask which allows for multiple functions to be performed simultaneously such as feeding and respiration.

US2005/0263150A1—Chatampally—Systems and Methods for the Administration of Drugs and Medications—discloses a nose and mouth mask which allows for delivery of drugs by respiration.

US2006/0231091A1—Camarillo—Non-Rebreathing Oxygen Face Mask With Nebulizer Attachment—discloses a face mask disposed with a nebulizer to avoid having to remove the mask to administer drugs.

US2008/0210242—Burk—Mask-Nebulizer-Metered-Dose-Inhaler Assembly—discloses a face mask disposed with a nebulizer and with a metered dose inhaler.

US2008/0245370A1—Kobziar—Oxygen Mask with Rebreather Bag for Use with Pulse Oxygen Delivery System—discloses a supplemental oxygen system with a rebreather bag in combination with a pulsed delivery system.

US2009/0250060A1—Hacke—Disposable Mask Assembly with Exhaust Filter and Valve Disc and Method of Assembling Same—discloses a facemask sized to fit a patient face, and a filter.

US2010/0116271A1—Horsford—Apparatus and Method for Simultaneous Delivery of Oxygen and Inhaled Medication—discloses an adapter to connect secondary devices and delivery to a primary face mask.

US2011/0190845—Weisfeld—Portable Negative Pressure Ventilation Device and Methods and Software Related Thereto—a system for stimulating the phrenic nerve to cause breathing by natural contraction of the diaphragm.

US2012/0216806A1—Rookard—Tube Ventilated Oxygen Mask—discloses methods and systems for improved oxygen flow through a standard non-rebreather oxygen mask.

US2013/0060157A1—Beard—Oxygen Facemask with Capnography Monitoring Ports—discloses an oxygen face mask with port for capnography.

US2013/0199595A1—Dhuper—Gas Delivery Venturi—discloses a system for controlling the concentration of gas delivered to face mask.

### BRIEF DESCRIPTION OF THE DRAWINGS

FIG. 1 is a side view diagram showing the empty elastic reservoir bag.

FIG. 2 is a side view diagram showing the elastic reservoir bag in a partially inflated state.

FIG. 3 is a side view diagram showing the elastic reservoir bag in a fully inflated state.

FIG. 4 is a frontal view diagram showing a standard face mask with one way check valves and a gasket for attachment of the elastic reservoir bag.

FIG. 5 is a frontal view diagram showing a standard face mask attached at the gasket to the elastic reservoir bag.

FIG. 6 is a front view diagram showing the improved face mask with an inflatable inner lining as well as the gasket and hose for inflating said lining.

FIG. 7 is a side view diagram showing the improved face mask with an inflatable inner lining.

### SUMMARY OF THE INVENTION

The invention provides the easy and simple use of a non-rebreather face mask.

An object of the present invention is to provide oxygen at a purity level of 80% or greater.

Another object of the present invention is to provide a slight amount of positive pressure on inhaling

Another object of the present invention is to improve the fit of face masks on the faces of a variety of different patients.

Another object of the present invention is to improve the fit of face masks on a variety of patients without obstructing the nostrils or mouth of any patient.

Another object of the present invention is to reduce dead space within the mask.

Another object of the present invention is to reduce the amount of pure oxygen that is required to run the system.

Another object of the present invention is to provide a non-rebreather face mask with additional safety features.

Various improvements of the present invention may be apparent to those skilled in the art.

### DETAILED DESCRIPTION OF THE DRAWINGS

Referring now to the drawings wherein the showings are for purposes of illustrating a preferred embodiment of the present invention and not for purposes of limiting the same.

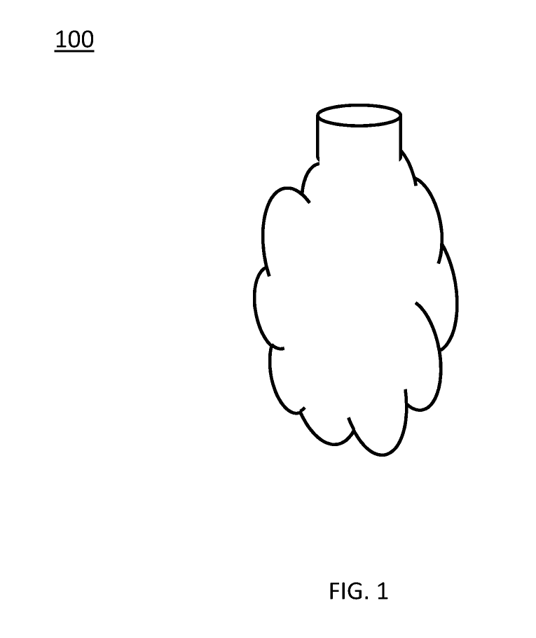

FIG. 1 shows an uninflated reservoir bag 100. The system is disposed with an elastic reservoir bag which may expand to a greater volume when sufficient pressure is supplied.

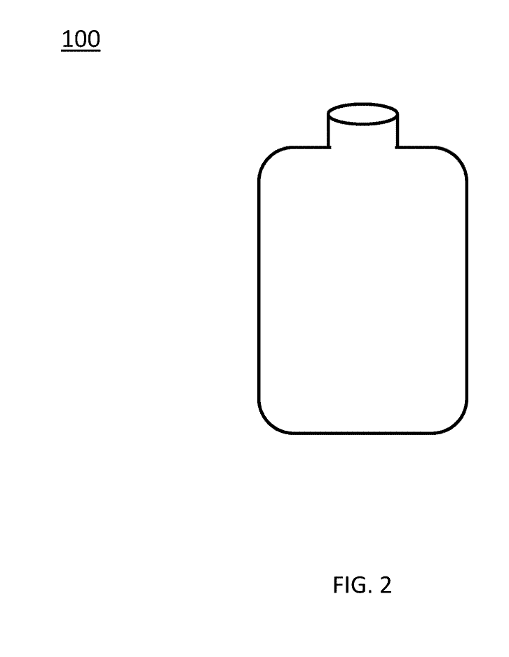

FIG. 2 shows a partially inflated reservoir bag 100. The bag 100 is semi-compliant so that a certain pressure will be sufficient to expand the bag to a working volume. The elastic reservoir bag provides a mild positive pressure to the patient's lungs and this increases lung compliance as well as increases the blood oxygen concentration of patients. The bag is resistant to any further expansion after that pressure is reached until a second threshold pressure is reached. Although the bag 100 could also be made of material to be substantially compliant with Boyles Law, that is that the volume is proportionally related to the pressure.

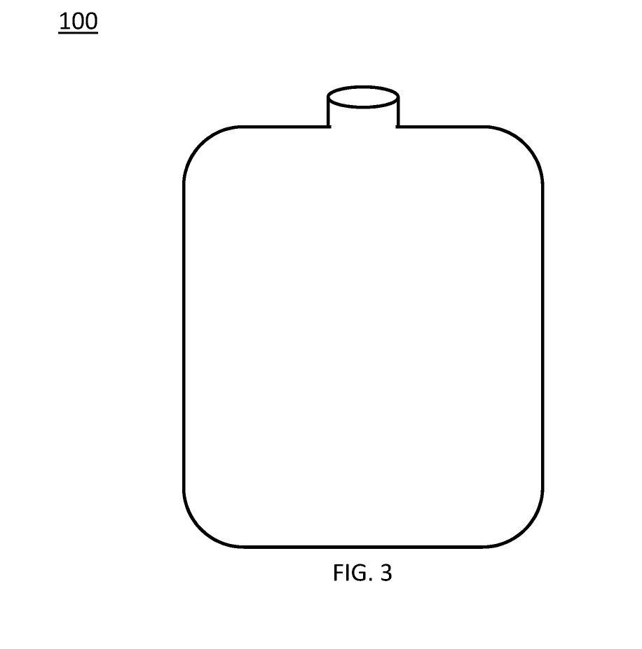

FIG. 3 shows a reservoir bag 100 that is fully inflated. Once a second threshold pressure is reached the reservoir bag is capable of a second expansion in volume. The bag may be constructed from plastic, rubber, silicon, or other surgical grade materials.

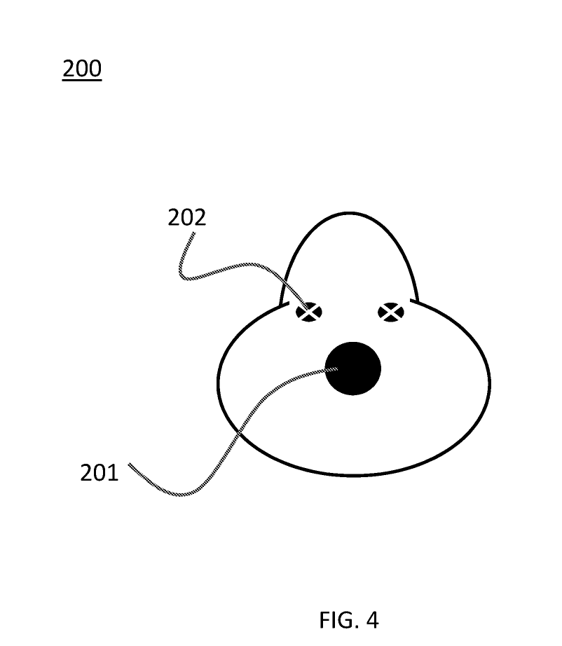

FIG. 4 shows a standard face mask 200 designed to cover both the nose and mouth of a patient. The proximal side of the mask is in contact with the patient's face. The distal side of the mask faces outwards away from the patient's face. The center of the distal side of the mask is disposed with a luer-lock 201 for attaching an elastic reservoir bag to the face mask (not shown). Adjacent to the luer-lock are two one-way check valves 202 which allow exhaled air to escape from the mask but prevent atmospheric air from entering the mask. The mask may be constructed from plastic, rubber, silicon, or other surgical grade materials.

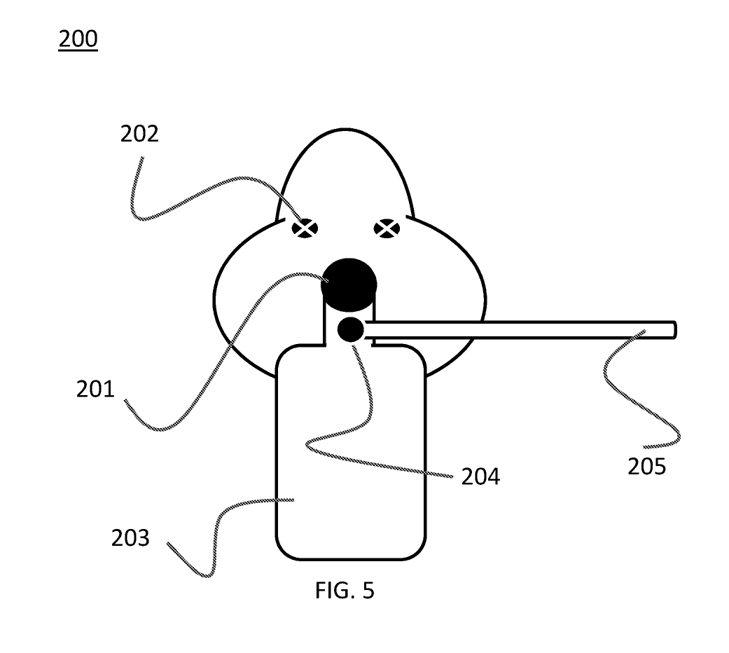

FIG. 5 shows a standard face mask 200 with an elastic reservoir bag 203 attached. The elastic reservoir bag 203 is further disposed with a luer-lock 204 for attachment of a hose 205 that connects to the oxygen source.

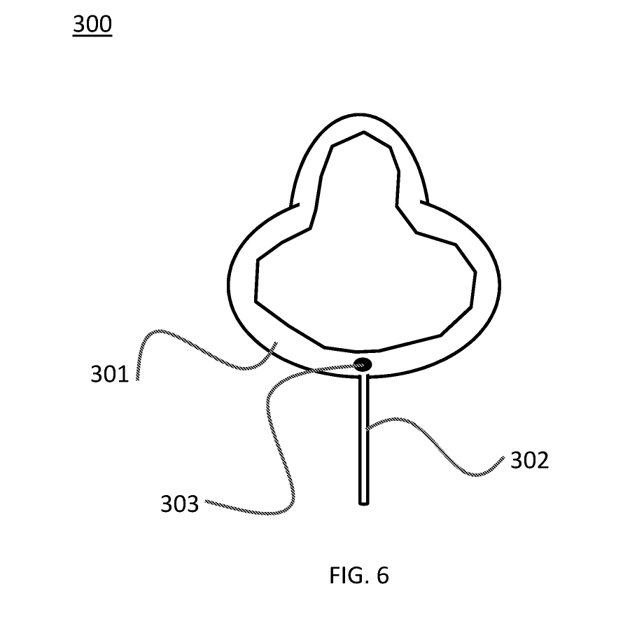

FIG. 6 shows an improved face mask 300 designed to fit upon the patient's face in an air-tight or near air-tight fashion and reduces dead space on the inner volume of the mask. This increase in tightness and dead space reduction is achieved by a soft compliant balloon 301 that lines the outermost edge of the face mask on the proximal, inner surface. In this embodiment the balloon 301 is disposed around the circumference of the face mask, but in another embodiment could be disposed as two areas that span the bridge of the nose similar in shape to saddlebags. In both embodiments the balloon 301 increases the tightness of fit and decreases dead space without obstructing the patient's nostril or mouth. The balloon 301 could be in any conformation that allows the dead space to be reduced and still allow the patient to inhale and exhale. The balloon 301 is connected to an air source for inflation by a tube 302 connected to a standard valve 303 disposed upon the lower, center edge of the mask just beneath the patient's mouth. Pressure levels inside the balloon may reach up to two atmospheres but is one and a half atmospheres in a preferred embodiment. Depending upon the patient receiving oxygen, the volume of the balloon 301 may vary between 25 milliliters for children to 100 milliliters for adults engaged in vigorous activity. The reduced dead space allows the system to be more efficient, requiring less pure oxygen to operate while supplying said oxygen at a higher concentration to the patient than most masks are capable of.

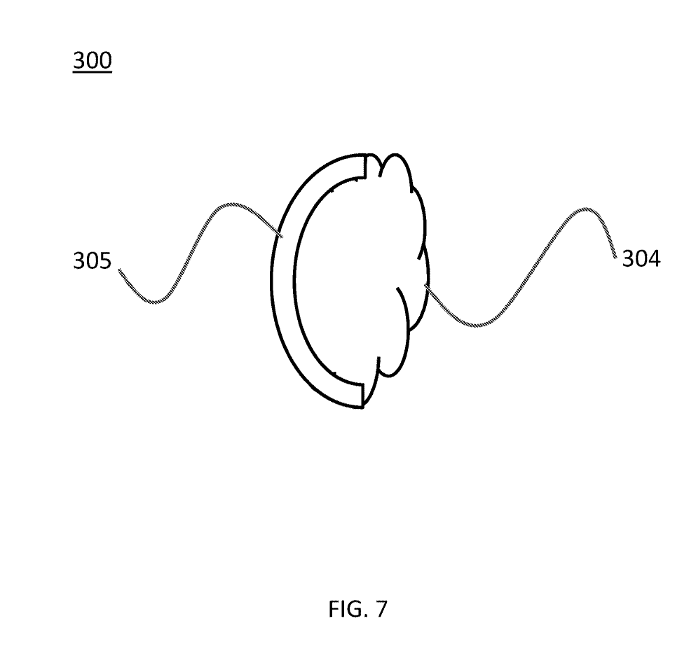

FIG. 7 shows an improved face mask 300 from a side view. This view shows the proximal side of the mask 304 facing the patient, the distal side of the mask 305 facing away from the patient, extent of balloon inflation and the reduction of dead space.

In another embodiment, the improved face mask may be sized to fit a child. Since children breathe at an average rate of 12-18 breaths per minute, the apparatus is disposed to provide pure O2 at the rate of 6-12 liters/min.

In another embodiment, the improved face mask may be adapted to suit seniors or people in the hospital. Since seniors and hospitalized people often breathe at a lower rate of about 12-18 breaths per minute, the apparatus is disposed to provide pure O2 at the rate of Y liters/min.

In another embodiment, the improved face mask may be adapted to suit a person breathing heavily while working out or exercising. In this embodiment the elastic reservoir bag is disposed with a 2-3 liter capacity and the apparatus is disposed to provide pure O2 at the rate of Y L/min to accommodate a work out breathe rate of 18-30 breaths per minute.

In another embodiment, the improved face mask may be adapted to allow a user to manually pump oxygen into the face mask.

In yet another embodiment, the improved face mask is disposed with a line connected directly to an oxygen source, without any intervening elastic reservoir bag. Typically such systems deliver oxygen between 28-50% purity but in this embodiment will delivery oxygen that is 50% pure or greater.

Additional modifications and improvements of the present invention may also be apparent to those skilled in the art. Thus, the particular combination of parts described and illustrated herein is intended to represent only one embodiment of the invention, and is not intended to serve as a limitation of alternative devices within the spirit and scope of the invention.

What is claimed:

1. A non-rebreather face mask for oxygen supplementation comprising:
A face mask with a balloon disposed along the circumference of the inner surface of the mask, that may be inflated to a volume of between 10-100 milliliters, further disposed with a standard valve at the center bottom of the mask for inflation by a tube connected to an external gas source, further disposed with two one way check valves for releasing gas from within the mask, further disposed with a luer lock for attachment to a semi-compliant elastic reservoir bag, which may be 0.1-5 liters, which is further disposed with a luer lock for attachment to a hose connected to a source of pure oxygen.

2. A non-rebreather face mask for oxygen supplementation comprising:
An elastic reservoir bag, a face mask with an inflatable inner lining, one way CO2 check valves, an O2 line.

3. A non-rebreather face mask for oxygen supplementation comprising:
a face mask with a reservoir bag attached to a bottom portion of the face mask, an oxygen line inlet on the reservoir bag for receiving oxygen, an exhaust valve for a user's exhaled breath, and a balloon attached to an inner surface of the face mask and wherein the balloon is inflated to occupy a dead space in the face mask but does not obstruct a user's nostril or mouth airways.

4. Wherein the balloon is incorporated into the circumference of the face mask in a generally circular configuration.

5. Wherein the balloon is configured in the shape of two saddle bags that rest over the user's nose and onto the user's cheeks.

6. Wherein the balloon is inflatable with a luer lock configured port on the face mask.

7. Wherein the balloon has a volume of between 10-25 milliliters.

8. Wherein the balloon has a volume of 25-50 milliliters.

9. Wherein the balloon has a volume of 50-100 milliliters.
